(19) United States
(12) Patent Application Publication
(10) Pub. No.: US 2015/0335497 A1
(43) Pub. Date: Nov. 26, 2015

(54) Cupcakes Nursing Pads

(71) Applicant: Cake Lingerie Pty Ltd, Castle Hill (AU)

(72) Inventor: Tracey Montford, Castle Hill NSW (AU)

(21) Appl. No.: 14/287,082

(22) Filed: May 26, 2014

(51) Int. Cl. A61F 13/505

(52) U.S. Cl. CPC A61F 13/505; A61F 2013/15016

(57) ABSTRACT

In the preferred forms, the invention provides a single purchase, nursing accessory that can be used for a multitude of needs depending on the milk flow at different times of the day or night as well at selected time schedules. The present invention also provides an increased ease with washing due to faster drying time because of the ability of the inserted layers being removable prior to washing. This also prevents the build up of bacteria that occur. The binding edge of the pad ensures the pad is durable as well as delivering the tailored result that many women prefer.

The present invention relates to breast or nursing pads.

### BACKGROUND OF THE INVENTION

Any discussion of the prior art throughout the specification should in no way be considered as an admission that such prior art is widely known or forms part of common general knowledge in the field.

Lactating women will feed their newborns several times a day. Suckling by the baby creates pressure and stimulates the breast to secrete milk. Unwanted leakage is very common amongst lactating women and the flow can vary depending on several factors and can be triggered by various stimuli.

The time of the day or night, the woman's body and the amount the baby tends to feed are some of the many factors that can determine excess flow and unwanted leakage from the breast.

A breast pad is a protective layer and a must have for lactating women. Breast pads are placed around the nipple area and on the inside of nursing bras to form a barrier which protects their bras and outer clothing from getting wet and stained due to the leaking of breast milk from the breast during non-feeding times.

There are typically 3 types of washable breast pads in the market—light flow, medium flow and heavy flow. The difference between the 3 types of nursing pads is its mid layer, which is thickest in the heavy flow and thus provides a greater capacity to absorb and retain excess milk and flow. Women will probably require all 3 types of these nursing pads depending on their level of leakage at various times.

In a conventional nursing pad, the pad consists of three key layers and these are all sewn together at the circumference edge that binds them together. The inner layer is in cotton, followed by a mid layer, which is an absorbent fabric, followed by an outer laminated layer of PUL and cotton which stops the moisture from wetting the clothing provided the nursing pad doesn't become too wet. Once wet the entire nursing pad needs to be replaced and washed.

Women generally carry spare nursing pads to change as they become wet. This avoids her bra and outwear from becoming wet and avoids the discomfort felt from wearing a wet nursing pad.

### BRIEF DESCRIPTION OF THE DRAWINGS

FIG. 1 is a front view of one embodiment of a breast pad consistent with the invention.

FIG. 2 is a cross-sectional view of the breast pad of FIG. 1.

FIG. 3 is a front view of a material used for an outer layer of the breast pad of FIG. 1.

FIG. 4 is a front view of a material and construction used for an inner layer of the breast pad of FIG. 1.

FIG. 5 is a front view of components used for the inner layer of FIG. 4.

FIGS. 6 and 7 are respective perspective and front views of a mid layer disc of the breast pad of FIG. 1.

### DETAILED DESCRIPTION

In preferred forms, the invention provides a single purchase, nursing accessory that can be used for a multitude of needs depending on the milk flow at different times of the day or night as well at selected time schedules. The present invention also provides an increased ease with washing due to faster drying time because of the ability of the inserted layers being removable prior to washing. This also prevents the build up of bacteria that occur. The binding edge of the pad ensures the pad is durable as well as delivering the tailored result that many women prefer.

The inner layer and the outer layer of the nursing pads may be sewn together at the circumference edge that binds them together. A pocket may be created on the inner layer through which mid-layer fabric discs, also referred to herein as pad inserts, can be added as required. The advantage of adding these discs is to increase the thickness of the nursing pad and therefore an increase in the level of absorbency.

The unique construction of this invention enables women to easily manage the effect of leakage by choosing to add the absorbent discs into the one nursing pad as opposed to having different nursing pads for different flows. Women are able to separately purchase the washable and absorbent discs and then separately choose to insert the discs into the nursing pad as needed. This is preferable to having to purchase and carry 3 different types of nursing pads due to not knowing which one will be needed for use.

The discs may be made from an absorbent hemp fabric to assist with the absorbency of excessive milk flow without the need to carry multiple nursing pads as required depending on the milk-flow level. This fabric disc construction enables the wearer to remove the inserted absorbent disc and wash it as required. This method decreases the wash and dry period considerably.

The inner fabric may have a wicking capability to absorb and pass through the moisture to the mid layer (disc), which traps and stores the moisture. The more added mid layers (discs) the more absorbent the nursing pad becomes.

Fabrics for the invention may be organic and of natural fibers. The inner layer may be organic cotton followed by the hemp absorbent mid-layer, followed by the outer layer, which may be a PUL lamination to organic cotton. The circumference of the breast pad may be bound with fabric knitted, purpose elastic to prevent the occurrence of fraying. The durability of the edge may be superior. Fabrics used may be selected to inhibit the growth of bacteria.

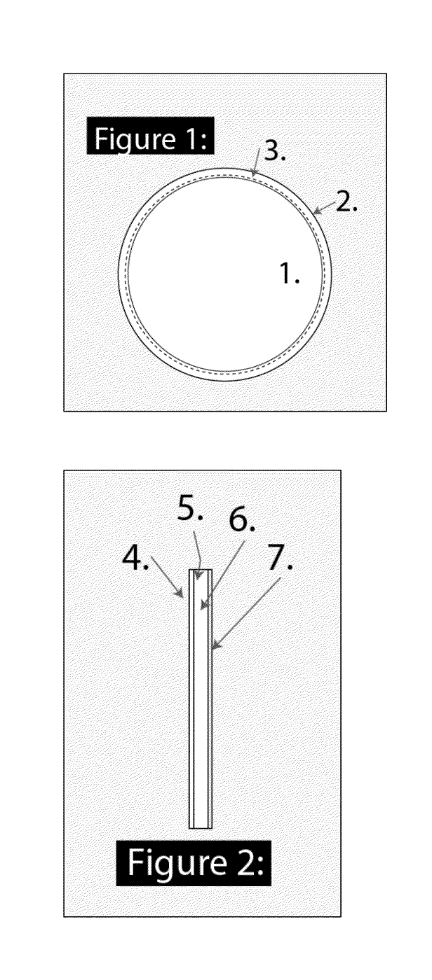

Now turning to FIGS. 1-7, FIG. 1 shows the outer face 1 of the breast pad for one embodiment of the invention. Outer face 1 includes a binding fold-over elastic 2 applied around the circumference of the breast pad using a single needle chain stitch 3.

FIG. 2 next shows a cross section of the breast pad layers construction, where an outer layer 4, formed of 100% organic cotton, is laminated to a layer 5 of Polyurethane Laminate (PUL), with an inner layer 7 disposed on an opposite face from outer layer 4 and defining a space 6 for an inserted pad. Inner layer 7 may be formed of cotton and may form an opening at a chord section across the inside layer of the pad.

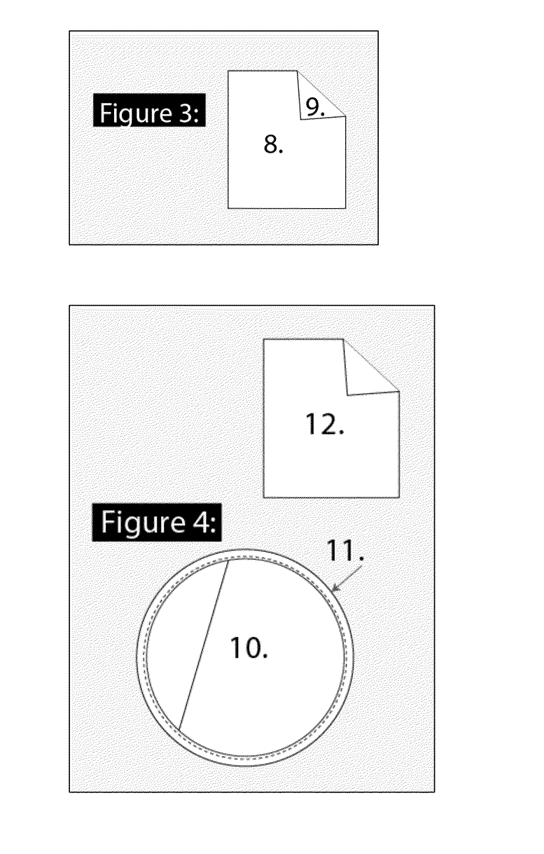

FIG. 3 next shows the fabric that the outer layer 4 (FIG. 2) may be constructed from, where face 8 of the outer fabric may be 100% organic cotton and face 9 of the outer fabric may be PUL.

FIG. 4 next shows the inner layer 7 (FIG. 2) construction and material. An inner face 10 may be constructed using 100% organic cotton with a folded edge along the chord of the circle forming the pad. In one embodiment, and as illustrated at 11, a fold-over binding elastic may be applied with a single needle chain-stitch application with a finished width of 6 mm. As illustrated at 12, the fabric that the inner layer is constructed from may be 100% organic cotton jersey knit.

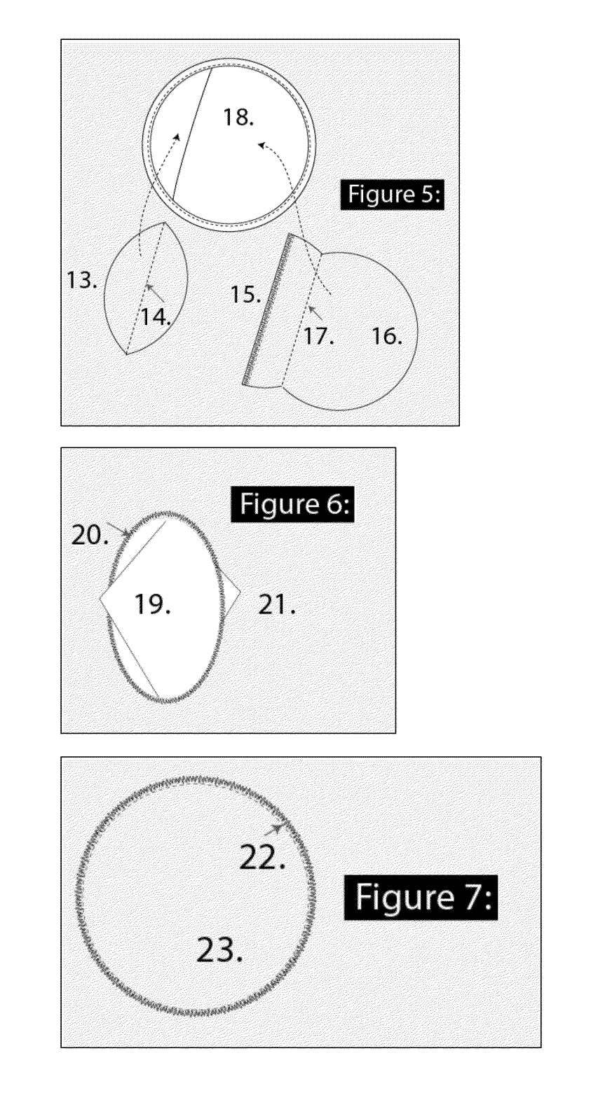

FIG. 5 illustrates an inner layer 7 (FIG. 2) construction with pattern piece formation. In particular, FIG. 5 illustrates a smaller chord section pattern piece 13 that may be formed of 100% organic cotton jersey, and which may be folded along folded line 14 to permit flexing for an opening function. A larger chord section pattern piece 16 that may be formed of 100% organic cotton jersey may be folded along folded line 17 to create an internally hidden over-locked edge 15 of the larger chord section pattern piece 16. It will be appreciated that lines 14 and 17 may generally meet one another to form an opening for receiving pad inserts, and thereby define a pocket 18 on the inner face of the breast pad.

FIG. 6 next illustrates from a side angle a front layer 19 of an absorbent mid layer disc or pad insert that may be made from hemp, and that is overlocked as illustrated at 20 with an opposing side 21 of the mid layer disc. As further illustrated by FIG. 7, the opposing side of the mid layer disc may also be formed of an absorbent hemp fabric layer 23 and overlocked along an overlocking edge 22.

It will be appreciated that while there are numerous ways in which the invention can be created the preferred method is exemplified in the figures above. This method offers a simple yet secure opening that remains tailored and is also comfortable.

In addition, by having a simple opening the structure allows itself, with comfort, to aesthetically incorporate ornate designs and prints to provide modified bindings and different pads. These can be made from a variety of fibers. The opening of the breast pad can be at the side of the section of the circumference or can use the diameter.

Although the invention has been described with reference to specific examples it will be appreciated by those skilled in the art that the invention may be embodied in many other forms.

1.-3. (canceled)

4. A breast pad, comprising:
first and second layers bound together substantially along a circumference edge and defining a pocket therebetween, the pocket having an opening that provides access to the pocket; and
an absorbent pad insert removably received in the pocket.

5. The breast pad of claim 4, wherein the first layer comprises an inner layer defining an inner face and the second layer comprises an outer layer defining an outer face.

6. The breast pad of claim 4, wherein the first layer defines the opening of the pocket along a chord section extending across the first layer.

7. The breast pad of claim 6, wherein the first and second layers are substantially circular in shape, and wherein the first layer includes first and second pattern pieces, each of the first and second pattern pieces folded along a folded line, and the first and second pattern pieces bound along the circumference edge such that the folded lines of the first and second pattern pieces extend across from one another along the chord section.

8. The breast pad of claim 4, further comprising a binding fold-over elastic extending along the circumference edge and bound together with the first and second layers.

9. The breast pad of claim 4, wherein the first layer is formed of a material with wicking capability to absorb and pass through moisture to the absorbent pad.

10. The breast pad of claim 9, wherein the first layer is formed of cotton.

11. The breast pad of claim 10, wherein the first layer is formed of jersey knit.

12. The breast pad of claim 4, wherein the second layer is formed of a laminated material.

13. The breast pad of claim 12, wherein the laminated material includes polyurethane laminate (PUL).

14. The breast pad of claim 13, wherein the laminated material further includes cotton laminated to the PUL.

15. The breast pad of claim 4, wherein the absorbent pad insert is a first absorbent pad insert, the breast pad further comprising a second absorbent pad insert removably received in the pocket along with the first absorbent pad insert.

16. The breast pad of claim 4, wherein the absorbent pad insert comprises hemp.

17. The breast pad of claim 16, wherein the absorbent pad insert is overlocked.

18. The breast pad of claim 4, wherein the pocket is configured to receive a varying number of absorbent pad inserts to vary a level of absorbency for the breast pad.

19. A breast pad, comprising:
first and second layers bound together substantially along a circumference edge and defining a pocket therebetween, the pocket having an opening that provides access to the pocket; and
a plurality of absorbent pad inserts configured to be removably received in the pocket, wherein the pocket is configured to receive a varying number of absorbent pad inserts among the plurality of absorbent pad inserts to vary a level of absorbency for the breast pad.

20. A method of configuring a breast pad, the method comprising:
selecting a number of absorbent pad inserts based upon a desired level of absorbency for the breast pad; and
inserting the selected number of absorbent pad inserts through an opening of a pocket defined between first and second layers of the breast pad that are bound together substantially along a circumference edge such that the selected number of absorbent pad inserts are removably received in the pocket.

21. The method of claim 20, further comprising increasing the level of absorbency for the breast pad by inserting an additional absorbent pad insert through the opening of the pocket.

22. The method of claim 20, further comprising decreasing the level of absorbency for the breast pad by removing at least one absorbent pad insert from the pocket through the opening.

23. The method of claim 20, further comprising:
removing at least one absorbent pad insert from the pocket through the opening;
washing the removed at least one absorbent pad insert; and
reinserting the washed at least one absorbent pad insert through the opening of the pocket.
